(19) United States
(12) Patent Application Publication
(10) Pub. No.: US 2024/0318551 A1
(43) Pub. Date: Sep. 26, 2024

(54) RESERVOIR FLUID TYPING

(71) Applicant: Equinor Energy AS, Stavanger (NO)

(72) Inventors: Alexandra CELY, Sandnes (NO); Tao YANG, Stavanger (NO); Ibnu Hafidz ARIEF, Oslo (NO); Gulnar YERKINKYZY, Sandvika (NO); Knut ULEBERG, Sandnes (NO)

(73) Assignee: Equinor Energy AS, Stavanger (NO)

(21) Appl. No.: 18/576,220

(22) PCT Filed: Jul. 15, 2022
(86) PCT No.: PCT/NO2022/050180

§ 371 Date: Jan. 3, 2024

(30) Foreign Application Priority Data
Jul. 15, 2021 (GB) 2110203.3

(51) Int. Cl. E21B 49/00; E21B 7/04; E21B 44/00; E21B 49/08

(52) U.S. Cl. CPC E21B 49/005; E21B 7/04; E21B 44/00; E21B 49/0875

(57) ABSTRACT

The geochemical parameters of reservoir fluid do not directly and universally correlate with the fluid type of the reservoir fluid, e.g. reservoir oil and reservoir gas. However, within an individual hydrocarbon basin, the local reservoir oils and the local reservoir gases are often geochemically distinct. Therefore, by examining various geochemical parameters for reservoir fluid samples taken from a particular region of interest, it is possible to identify region-specific thresholds for those geochemical parameters, and also to identify particular region-specific thresholds having a high degree of confidence for distinguishing between different reservoir fluid types. Advantageously, many geochemical parameters can be determined using mud-gas data, and in some cases using only standard mud-gas data. Therefore, by collecting mud-gas data when drilling a new well within the region of interest, these region-specific thresholds can be used to generate a substantially continuous and highly accurate reservoir fluid type log along a length of the well. This same technique may also be applied retrospectively to existing wells where mud-gas data was collected at the time of drilling, since at least standard mud-gas data is routinely collected while drilling.

The present invention relates to a system and method for identifying the fluid type of a target reservoir fluid.

Drilling fluid is a fluid used to aid the drilling of boreholes into the earth. The main functions of drilling fluid include providing hydrostatic pressure to prevent formation fluids from entering into the well bore, keeping the drill bit cool and clean during drilling, carrying out drill cuttings, and suspending the drill cuttings while drilling is paused and when the drilling assembly is brought in and out of the hole. Drilling fluids are broadly categorised into water-based drilling fluid, non-aqueous drilling fluid, often referred to as oil-based drilling fluid, and gaseous drilling fluid. Liquid drilling fluids, i.e. water-based drilling fluid or non-aqueous drilling fluid, are commonly referred to as “drilling mud”.

Mud-gas logging entails gathering data from hydrocarbon gas detectors that record the levels of gases brought up to the surface in the drilling mud during a bore drilling operation.

The most common gas component present is usually methane (C₁). The presence of intermediate hydrocarbons, such as C₂ (ethane), C₃ (propane), C₄ (butane) and C₅ (pentane) may indicate an oil or a “wet” gas zone. Heavier molecules, up to about C₈ (octane), may also be detectable, but are typically present only in very low concentrations. Consequently, the concentrations of these hydrocarbons are often not recorded.

There are two types of mud-gas data that can be collected, which are sometimes referred to a “standard” mud-gas logging, and “advanced” mud-gas logging. The equipment for standard mud gas logging and advanced mud gas logging are different.

For a standard mud gas system, the degasser does not usually have heating or use constant volume gas separation. There is also only one mud sampling point (“out”) and therefore it is not suitable for recycling correction. The measured gas composition is usually referred to as standard mud-gas data, which is not directly comparable to the actual C₁ to C₅ composition of the reservoir fluid sample.

For an advanced mud gas system, the degasser has heating and usually uses a constant volume for gas separation. There are two sampling mud points (“out” and “in”), and therefore it is possible to perform recycling correction. The measured gas composition is usually referred advanced mud-gas data.

When generating advanced mud-gas data, in order to make the data closely correspond to the actual reservoir fluid C₁ to C₅ concentrations, two correction processes are applied to the “raw” mud-gas data from the advanced mud gas logging system.

Firstly, a recycling correction is made to eliminate contamination of the gas by gases originating from previous injections of the drilling mud. This correction is applied based on a separate mud-gas measurement that was taken before the drilling mud was injected into the drilling string.

Secondly, an extraction efficiency correction step is applied to increase the concentration of intermediate components (from C₂ to C₅), such that the concentration of these components, relative to the C₁ concentration, more closely resemble the relative compositions of a corresponding reservoir fluid sample. The extraction efficiency correction is applied based on the type of drilling mud used for the borehole.

In the past, the advanced mud gas data has been examined to estimate certain fluid properties of the reservoir fluid using broad, empirical correlations between the advanced mud-gas composition and certain fluid properties of the reservoir fluid. For example, extremely dry gas reservoirs should comprise mostly C₁ and not much C₂₊, e.g. with C₁/C₂ ratios being greater than 50. Wet gas reservoirs will often have ratios between 15 and 50, and oil reservoirs will have ratios between 2 and 15.

These empirical corrections are known to be highly inaccurate, particularly close to the range boundaries, and so were rarely relied upon in isolation, but rather were used to guide where to take downhole fluid samples. However, more recently, an advanced machine learning model has been developed that has made it possible to predict reservoir fluid properties much more accurately from the advanced mud-gas data.

Details of how such a machine learning model was trained to determine a gas-oil ratio of the reservoir fluid based on the advanced mud-gas data can be found in the paper Tao Yang et. al. (2019), “A Machine Learning Approach to Predict Gas Oil Ratio Based on Advanced Mud Gas Data”. Society of Petroleum Engineers. doi: 10.2118/195459-MS

Advantageously, this model can be used to generate a substantially continuous log of the respective reservoir fluid property. This was not previously possible, and in the past, it was necessary to rely on downhole fluid samples taken at discrete intervals. Furthermore, the model allows reservoir fluid property predictions to be made at a very early stage of the drilling process and without needing to interrupt the drilling process, as might be required to take downhole fluid samples or the like.

This model has been found to be very useful, but is limited in that it requires the availability of advanced mud-gas data. Whilst the collection of advanced mud-gas data is less costly than some techniques, such as downhole fluid sampling, it still adds additional costs to the drilling process.

A need therefore exists for a new technique for identifying properties of a reservoir fluid.

Viewed from a first aspect, the present invention provides a method of identifying a fluid type of a target reservoir fluid within a region of interest, comprising: obtaining reservoir fluid properties data corresponding to a plurality of fluid samples; identifying a fluid type and at least one geochemical parameter for each of the fluid samples that are within the region of interest, the or each geochemical parameter being derivable from C₁ to C₅ fluid composition data; determining a region-specific threshold for the or each geochemical parameter based on the fluid type of the plurality of fluid samples within the region of interest, the region-specific threshold being for distinguishing between a first fluid type and a second fluid type within the region of interest; obtaining mud-gas data from a well drilled through the target reservoir fluid; identifying at least one geochemical parameter for the target reservoir fluid based on the mud-gas data; and identifying a fluid type of the target reservoir fluid based on the region-specific threshold associated with the or each of the at least one geochemical parameter for the target reservoir fluid.

The present inventors have recognised that the geochemical parameters of reservoir fluid do not directly and universally correlate with the fluid type of the reservoir fluid, e.g. reservoir oil and reservoir gas. However, within a particular, geographic region, the local reservoir oils and the local reservoir gases are often geochemically distinct. Therefore, by examining various geochemical parameters for reservoir fluid samples taken from the particular region of interest, it is possible to identify region-specific thresholds for those geochemical parameters.

Advantageously, where the geochemical parameters can be determined using mud-gas data, these region-specific thresholds can be used to rapidly, accurately and cheaply determine the fluid type of a target reservoir fluid. This technique may not provide detailed reservoir fluid property data, such as can be obtained other techniques, such as the machine-learning technique discussed above. However, accurately identifying the fluid type of a reservoir fluid is often sufficient for many early-stage decisions, such as geosteering during well drilling and casing perforation during well completion.

In various examples, the at least one geochemical parameter comprises any one or more or all of the following geochemical parameters: a C₁/C₂ ratio; a Bernard parameter, C₁/(C₂+C₃); a balance ratio, (C₁+C₂)/(C₃+C₄+C₅); a wetness ratio, (C₂+C₃+C₄+C₅)/(C₁+C₂+C₃+C₄+C₅); a dryness ratio, C₁/(C₁+C₂+C₃+C₄+C₅); and a hydrocarbon character, (C₄+C₅)/(C₃). However, any suitable geochemical parameter may be used, so long as it is derivable from mud-gas data. Thus, the at least one geochemical parameter may additionally or alternative comprise other geochemical parameters.

The method may further comprise determining a threshold confidence for the region-specific threshold associated with the or each geochemical parameter. The threshold confidence may be determined based on the fluid samples that are within the region of interest, and particularly based upon the fluid type and the respective geochemical parameter of each of the fluid samples that are within the region of interest. The threshold confidence may be determined using any suitable statistical method.

The threshold confidence may be indicative of a confidence associated with the region-specific threshold for distinguishing between the first fluid type and the second fluid type within the region of interest. That is to say, a confidence that a corresponding geochemical parameter value for a fluid sample from the region of interest that is below the region-specific threshold will correspond to one of the fluid types, and one that is above the region-specific threshold will correspond to the other of the fluid types.

The threshold confidences may be useful to informing an operator regarding the accuracy of a particular fluid type determination. Furthermore, it may indicate which of the geochemical parameters should be used for a particular region of interest, as not all parameter may provide sufficient accuracy when determining the fluid type.

Whilst the method may be employed using a single geochemical parameter, preferably the one or more geochemical parameter comprises a plurality of geochemical parameters.

The method may further comprise identifying at least one distinguishing geochemical parameter from amongst the one or more geochemical parameter based on the threshold confidences. The at least one distinguishing geochemical parameter may be region-specific, i.e. for distinguishing fluid types within the region of interest. Identifying the fluid type of the target reservoir fluid may be based on the region-specific threshold associated with the or each of the at least one distinguishing geochemical parameter for the target reservoir fluid.

The at least one distinguishing geochemical parameter is preferably a subset of the at least one geochemical parameter. The method may examine multiple geochemical parameters, and select a subset (optionally including all of them if appropriate) based on the threshold confidences. That this to say, the original geochemical parameters may be test geochemical parameters, which may be evaluated to determine the distinguishing geochemical parameters having sufficient confidence for the region of interest. Preferably, those test geochemical parameters having the highest confidences are selected, for example having a threshold confidence above a predetermined threshold.

Optionally, identifying the fluid type of the target reservoir fluid may be further based on a weighting based on the threshold confidences associated with the at least one geochemical parameter. For example, a fluid type indication based on a geochemical parameter having a relatively high confidence may be given greater weight than a fluid type indication based on a geochemical parameter having a relatively low confidence.

Additionally, the threshold confidences may be used to guide an operator regarding what data should be collected.

Obtaining the mud-gas data may comprise measuring mud-gas data as the well is drilled.

The method may comprise determining that a threshold confidence associated with a geochemical parameter derivable from C₁ to C₃ fluid composition data is above a predetermined level. Consequently, obtaining the mud-gas data may comprise obtaining standard mud gas data in response to the determination.

This may be advantageous, as standard mud-gas data is cheaper to collect that advanced mud-gas data.

The method may comprises determining that a threshold confidence associated with a geochemical parameter derivable from C₁ to C₃ fluid composition data is below a predetermined level. Consequently, obtaining the mud-gas data comprises obtaining advanced mud gas data and/or obtaining standard mud gas using heating in response to the determination. The heating may comprise heating to a temperature of at least 40° C., at least 50° C., at least 70° C., at least 80° C., or at least 90° C.

Using advanced mud gas data and/or heated standard mud gas improves the accuracy of C₄ and C₅ fluid composition data, which allows for a larger number of geochemical parameters to be used when those that can be calculated based on C₁ to C₃ fluid composition data are insufficient.

The first fluid type may be reservoir oil and the second fluid type may be reservoir gas.

The region of interest may comprise a single hydrocarbon field or a single hydrocarbon basin.

The plurality of fluid samples may comprise data from a plurality of regions, such as a plurality of hydrocarbon fields or hydrocarbon basins. The method may comprise identifying a subset of the plurality of fluid samples that are within the region of interest. The identification may be based on geophysical data associated with the fluid samples. Additionally, or alternatively, the identification may be based on the reservoir fluid properties data, and particularly based on identifying fluid samples having similar reservoir fluid properties data, which may indicate that they have been taken from the same hydrocarbon field or hydrocarbon basin.

The method may advantageously be applied to multiple target reservoir fluids. For example, the mud-gas data may comprise a plurality of mud-gas data points, which may correspond to target reservoir fluids at a plurality of locations along a length of the well. Identifying the fluid type may then comprise identifying a fluid type of the target reservoir fluid at each location along the length of the well. Thus, the method can be used to generate a fluid type log along a length of the well.

There are multiple possible applications for accurate fluid typing. In one example, it may be used during completion of a well. For example, the method may comprise determining a perforation location of a casing of the well based on the identified fluid type. The method may further comprise perforating the casing of the well at the determined location.

In another example, the fluid typing may be used to assist a drilling operator whilst drilling the well. For example, the method may comprise steering drilling of a well based on the fluid type of the target reservoir fluid. This is often known as geosteering.

In a preferred embodiment the method is a computer-implemented method, e.g. the steps of the method are performed by processing circuitry.

The method may be implemented at least partially using software, e.g. computer programs. It will thus be seen that when viewed from further aspects the present invention provides computer software specifically adapted to carry out the methods described herein when installed on a data processor, a computer program element comprising computer software code portions for performing the methods described herein when the program element is run on a data processor, and a computer program comprising code adapted to perform all the steps of a method or of the methods described herein when the program is run on a data processing system.

It will further be appreciated that not all steps of the methods need be carried out by computer software and thus from a further broad embodiment the present invention provides computer software and such software installed on a computer software carrier for carrying out at least one of the steps of the methods set out herein.

Any one or more of the following steps may be performed computer software: identifying a fluid type and at least one geochemical parameter for each of the fluid samples that are within the region of interest; determining the region-specific threshold; determining the threshold confidence; identifying the one or more geochemical parameter for the target reservoir fluid; and identifying a fluid type of the target reservoir fluid

According to one embodiment, obtaining the reservoir fluid properties data comprises reading the reservoir fluid properties data from a database; and/or obtaining the mud-gas data comprises reading the mud-gas data from a database or receiving data input by a user.

The present invention also extends to a computer software carrier comprising such software arranged to carry out the steps of the methods of the present invention. Such a computer software carrier could be a physical storage medium such as a ROM chip, CD ROM, DVD, RAM, flash memory or disk, or could be a signal such as an electronic signal over wires, an optical signal or a radio signal such as to a satellite or the like.

The present invention may accordingly suitably be embodied as a computer program product for use with a computer system. Such an implementation may comprise a series of computer readable instructions, which may be fixed on a tangible, non-transitory medium, such as a computer readable medium, for example, diskette, CD ROM, DVD, ROM, RAM, flash memory or hard disk. It could also comprise a series of computer readable instructions transmittable to a computer system, via a modem or other interface device, over either a tangible medium, including but not limited to optical or analogue communications lines, or intangibly using wireless techniques, including but not limited to microwave, infrared or other transmission techniques. The series of computer readable instructions embodies all or part of the functionality previously described herein.

Those skilled in the art will appreciate that such computer readable instructions can be written in a number of programming languages for use with many computer architectures or operating systems. Further, such instructions may be stored using any memory technology, present or future, including but not limited to, semiconductor, magnetic or optical, or transmitted using any communications technology, present or future, including but not limited to optical, infrared or microwave. It is contemplated that such a computer program product may be distributed as a removable medium with accompanying printed or electronic documentation, for example, shrink wrapped software, pre-loaded with a computer system, for example, on a system ROM or fixed disk, or distributed from a server or electronic bulletin board over a network, for example, the Internet or World Wide Web.

Thus, viewed from a second aspect, the present invention provides a computer program product or a tangible computer-readable medium storing a computer program product, the computer program product comprising computer readable instructions that, when executed by a computer, will cause the computer to perform a method comprising: obtaining reservoir fluid properties data corresponding to a plurality of fluid samples; identifying a fluid type and at least one geochemical parameter for each of the fluid samples that are within a region of interest, the or each geochemical parameter being derivable from C₁ to C₅ fluid composition data; determining a region-specific threshold for the or each geochemical parameter based on the fluid type of the plurality of fluid samples within the region of interest, the region-specific threshold being for distinguishing between a first fluid type and a second fluid type within the region of interest; obtaining mud-gas data from a well drilled through a target reservoir fluid; identifying at least one geochemical parameter for the target reservoir fluid based on the mud-gas data; and identifying a fluid type of the target reservoir fluid based on the region-specific threshold associated with the or each of the at least one geochemical parameter for the target reservoir fluid.

Viewed from a third aspect, the present invention provides a computer comprising: a memory, and a processor, the memory storing computer readable instructions that, when executed by the processor will perform a method comprising: obtaining reservoir fluid properties data corresponding to a plurality of fluid samples; identifying a fluid type and at least one geochemical parameter for each of the fluid samples that are within a region of interest, the or each geochemical parameter being derivable from C₁ to C₅ fluid composition data; determining a region-specific threshold for the or each geochemical parameter based on the fluid type of the plurality of fluid samples within the region of interest, the region-specific threshold being for distinguishing between a first fluid type and a second fluid type within the region of interest; obtaining mud-gas data from a well drilled through a target reservoir fluid; identifying at least one geochemical parameter for the target reservoir fluid based on the mud-gas data; and identifying a fluid type of the target reservoir fluid based on the region-specific threshold associated with the or each of the at least one geochemical parameter for the target reservoir fluid.

Certain preferred embodiments of the present disclosure will now be described in greater detail, by way of example only and with reference to the accompanying drawings, in which:

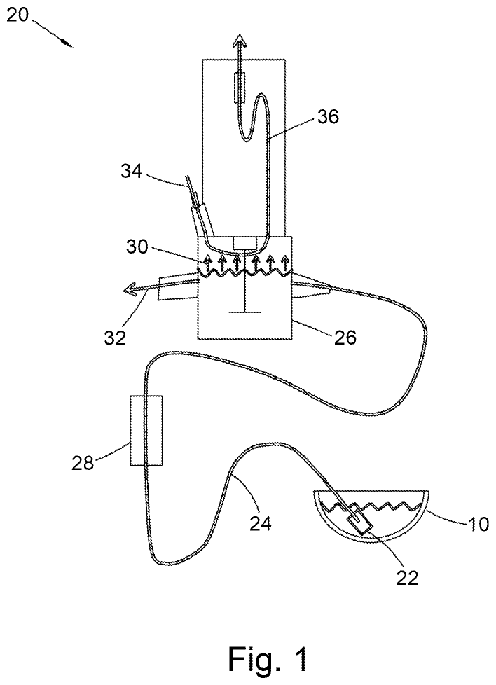

FIG. 1 is a schematic illustration of a mud-gas analysis tool;

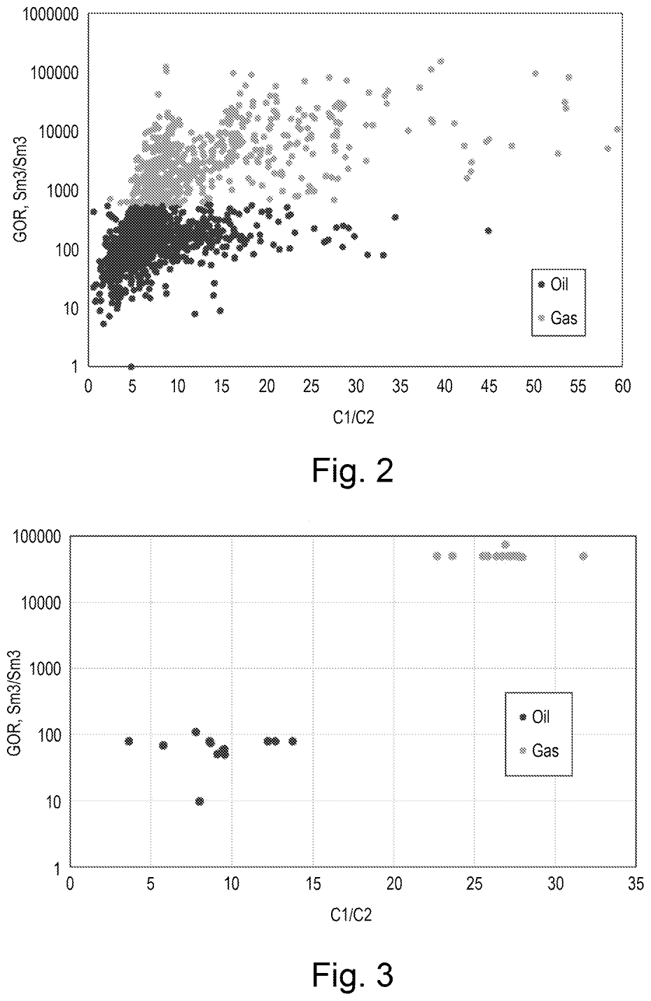

FIG. 2 shows a plot of gas-oil ratio against C₁/C₂ ratio for multiple fields;

FIG. 3 shows a plot of gas-oil ratio against C₁/C₂ ratio for a first field;

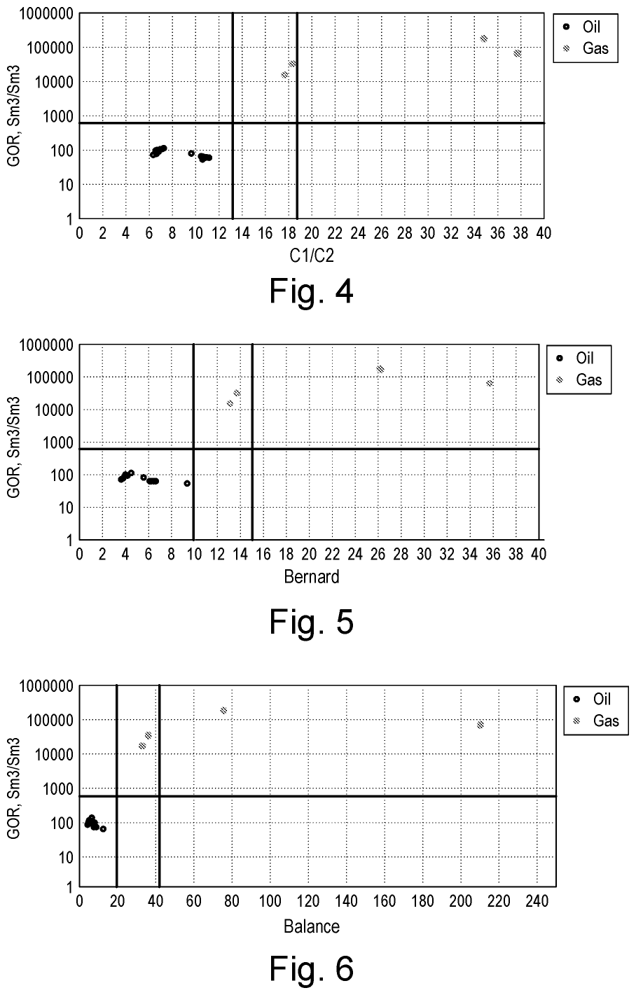

FIG. 4 shows a plot of gas-oil ratio against C₁/C₂ ratio for a second field;

FIG. 5 shows a plot of gas-oil ratio against Bernard parameter for the second field;

FIG. 6 shows a plot of gas-oil ratio against balance ratio (Bh) for the second field;

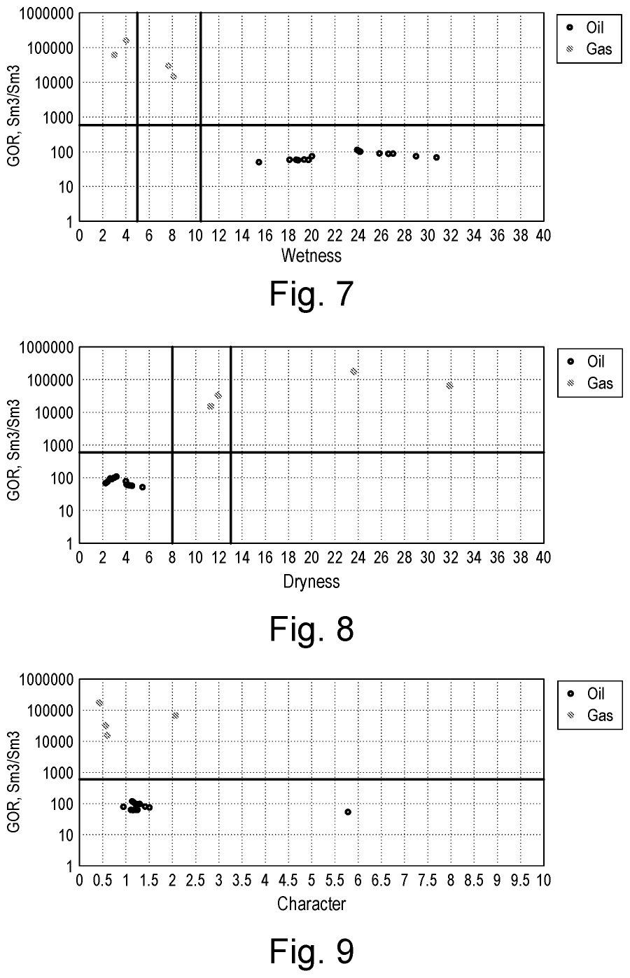

FIG. 7 shows a plot of gas-oil ratio against wetness (Wh) for the second field;

FIG. 8 shows a plot of gas-oil ratio against dryness for the second field; and

FIG. 9 shows a plot of gas-oil ratio against hydrocarbon character (Ch) for the second field.

An exemplary mud-gas analysis tool 20 is shown schematically in FIG. 1.

The tool 20 is coupled to a flow line 10 containing drilling mud returned from a borehole of a well. As discussed above, the drilling mud may be water-based mud or oil-based mud.

The tool 20 comprises a sampling probe 22 disposed with respect to the flow line 10 so as to collect a sample 24 of the drilling mud from the flow line 10. The drilling mud sample 24 is preferably a continuous sample, i.e. such that a portion of the flow of drilling mud within the flow line 10 is diverted through the mud-gas analysis tool 20.

The drilling mud sample 24 is supplied to a gas-separation chamber 26 where at least a portion of the gas carried by the drilling mud is released. The sample of drilling mud may optionally be heated by a heater 28 upstream of the gas-separation chamber 26. Heating the drilling mud sample 24 helps to release the gas from the drilling mud sample 24. Typically, for standard mud-gas data, the mud sample 24 is not heated with the temperature typically ranging from 10° C. to 60° C. However, in some implementations, heating is used to heat the drilling mud to around 80° C. to 90° C.

The released gas 30 is directed from the separation chamber 26 to a gas analysis unit (not shown), while the degassed mud 32 is returned to the flow line 10 or to another location for re-use.

The gas analyser may comprise a total gas detector, which may provide a basic quantitative indication as to how much gas is being extracted from the drilling mud by the tool 20. Total gas detection typically incorporates either a catalytic filament detector, also called a hotwire detector, or a hydrogen flame ionization detector.

A catalytic filament detector operates on the principle of catalytic combustion of hydrocarbons in the presence of a heated platinum wire at gas concentration below the lower explosive limit. The increasing heat due to combustion causes a corresponding increase in the resistance of the platinum wire filament. This resistance increase may be measured through the use of a Wheatstone bridge or equivalent detection circuit.

A hydrogen flame ionization detector functions on the principle of hydrocarbon molecule ionization in the presence of a very hot hydrogen flame. These ions are subjected to a strong electrical field resulting in a measurable current flow.

The gas analysis device may additionally or alternatively comprise an apparatus for detailed analysis of the hydrocarbon mixture. This analysis is usually performed by a gas chromatograph. However, several other detecting devices may also be utilised including a mass spectrometer, an infrared analyser or a thermal conductivity analyser.

A gas chromatograph is a rapid sampling, batch processing instrument that provides a proportional analysis of a series of hydrocarbons. Gas chromatographs can be configured to separate almost any suite of gases, but typically oilfield chromatographs are designed to separate the paraffin series of hydrocarbons from methane (C₁) through pentane (C₅) at room temperature, using air as a carrier. The chromatograph will report (in units or in mole percent) the quantity of each component of the gas detected.

A carrier gas stream 34, commonly comprising air, may be supplied to the separation chamber 26 and mixed with the released gas 30 to form a gas mixture 36 that is supplied to the gas analysis unit. The carrier gas stream 34 provides a continuous flow of carrier gas in order to provide a substantially continuous flow rate of the gas mixture 36 from separation chamber 26 to the gas analysis unit. Additionally, in the case of a gas analyser comprising a combustor, the use of air as the carrier gas may provide the necessary oxygen for combustion.

Commonly, mud-gas data provides a concentration for each of the C₁, C₂, C₃, iC₄, nC₄, iC₅, and nC₅ hydrocarbon gases.

In some arrangements, the tool 20 may be configured to detect and/or remove H₂S from the gas to prevent adverse effects that could influence hydrocarbon detection.

In some embodiments, non-combustibles gases, such as helium, carbon dioxide and nitrogen, can be detected by the gas analyser in conjunction with the logging of hydrocarbons.

Commonly, two standard mud-gas analysis tools 20 are permanently installed on drilling rigs. Both are connected to the drilling mud outlet, with one acting as a primary tool, and the other acting as a backup tool in case of failure of the primary tool. This ensures that at least standard mud-gas data can always be collected when drilling a well.

As discussed above, advanced mud-gas data is collected using different analysis tools. The main difference between a standard mud-gas analysis tool and an advanced mud-gas analysis tool is the use of a heater 28. However, there may also be other differences.

When it is desired to collect advanced mud-gas data for a particular well, two advanced mud-gas analysis tools are temporarily installed on the drilling rig, in addition to the permanent standard mud-gas analysis tools. One advanced mud-gas analysis tools is connected to the drilling mud inlet and the other is connected to the drilling mud outlet, so as to allow for collection of the two sets of data required for a recycling correction to be applied.

After drilling of a well where advanced mud-gas data has been collected, the advanced mud-gas analysis tools are removed.

In the past, the advanced mud gas data has been examined to estimate certain fluid properties of the reservoir fluid using broad, empirical correlations between the advanced mud-gas composition and certain fluid properties of the reservoir fluid. For example, extremely dry gas reservoirs should comprise mostly C₁ and not much C₂₊, e.g. with C₁/C₂ ratios being greater than 50. Wet gas reservoirs will often have ratios between 15 and 50, and oil reservoirs will have ratios between 2 and 15. However, these empirical corrections are known to be highly inaccurate, particularly close to the boundaries and so are rarely relied upon in isolation.

FIG. 2 shows a plot containing data from approximately 4000 reservoir fluid samples taken across a large range of oil fields around the world. The plot correlates a gas-oil ratio of the fluid sample (y-axis) against a C₁/C₂ ratio of the fluid sample. The term gas-oil ratio refers to the ratio of the volume of gas that comes out of solution at surface conditions to the volume of oil.

Green dots are used to indicate oil-phase fluid samples, and red dots are used to indicate gas-phase fluid samples. At reservoir conditions, the phase boundary between gas and oil occurs at a gas-oil ratio of approximately 600 Sm³/Sm³.

As will be apparent from FIG. 2, there is a significant overlap between gas and oil fluid samples in the C₁/C₂ ratio range of 5 to 20. Within this range, it is not possible to confidently distinguish between oil and gas fluid samples based only on the C₁/C₂ ratio. However, a large number of reservoirs have fluid compositions falling within this range, which is the reason why the previous empirical correlations cannot be relied upon.

Whilst only the C₁/C₂ ratio is shown here, other geochemical parameters can also be used to distinguish between gas phase and oil phase fluids, and similar empirical correlations exist for many of these parameters. However, they also have similar degrees of uncertainty. Such geochemical parameters include the Bernard parameter (C₁/C₂+C₃), the balance ratio (C₁+C₂/C₃+C₄+C₅), the wetness ratio (C₂+C₃+C₄+C₅/C₁+C₂+C₃+C₄+C₅), the dryness ratio (C₁/C₁+C₂+C₃+C₄+C₅), and the hydrocarbon character (C₄+C₅/C₃).

Prior to the development of the machine learning techniques described above, when advanced mud-gas data was to be examined, multiple geochemical parameters would be calculated and each compared to the respective empirical threshold. An estimation of whether a particular reservoir contains oil or gas would then be made based on what the majority of geochemical parameters indicated. This would typically achieve about 60% to 70% accuracy in identifying the reservoir fluid type. Whilst this provided a useful indicator, it was not sufficiently precise to be confidently relied upon.

The present inventors have examined the reservoir composition data and identified that although there is a great degree of overlap between geochemical parameters when considered globally, there is often a much clearer separation between the geochemical parameter values for gas and oil fluid samples within individual reservoirs.

Specifically, within a single hydrocarbon basin, the reservoir oils and gases are often from the same origin. Consequently, by considering only the specific oils and gases from a single basin, it is often possible to easily distinguish these specific fluids based on their geochemical parameters.

FIG. 3 illustrates 25 fluid samples taken from a first field. The oil samples are shown in green, and the gas samples are shown in red. As can be seen, in this particular reservoir, all of the oil samples have a C₁/C₂ ratio of below 14, whilst all of the gas samples have a C₁/C₂ ratio of greater than 22.

Based on this data, a region-specific C₁/C₂ ratio threshold can be determined for distinguishing between oil and gas within this reservoir, for example of about 18. This region-specific threshold can then be used when drilling new wells within the same field, similar to how the global thresholds were used in the past. However, significantly, a much greater confidence can be associated with that threshold, thereby allowing decisions to be made based purely on this geochemical analysis.

This is advantageous because many geochemical parameters can be determined based on mud-gas data collected whilst drilling the reservoir. This data is comparatively cheap to collect, is also available at a very early stage of well drilling, and can be collected as a continuous log along the length of the well. Thus, it can inform decisions in real-time regarding how to place and complete the well, such as where to perforate the well casing.

Whilst FIG. 3 shows only a plot for the C₁/C₂ ratio of this reservoir, this clear delineation between oil and gas fluid samples is also present for other geochemical parameters.

FIGS. 4 to 9 show a plot of gas-oil ratio against various geochemical parameters for 19 reservoir fluid samples taken from a second field. The figures show, respectively, the C₁/C₂ ratio, the Bernard parameter, the balance ratio, the wetness ratio, the dryness ratio, and the hydrocarbon character.

As can be seen, each of FIGS. 4 to 8 show a clear delineation between the gas samples and the oil samples. This indicates that a region-specific parameter threshold could be determined for any of the parameters shown in these figures (the C₁/C₂ ratio, the Bernard parameter, the balance ratio, the wetness ratio, the dryness ratio), and that these region-specific thresholds could be used to accurately distinguish between gas and oil reservoir fluids within this field.

FIG. 9 plots gas-oil ratio against the hydrocarbon character parameter, and for this reservoir there is a significant overlap between the hydrocarbon character parameters of the gas and oil samples. Consequently, whilst a region-specific threshold could be determined for this parameter, it would have a much lower confidence associated with it than the geochemical parameters plotted in FIGS. 4 to 8, and therefore would not be a good parameter to distinguish between oil and gas within this field.

Based on this knowledge, it is possible to identify not only more accurate, region-specific parameter thresholds for various geochemical parameters, but also to identify a confidence associated with the thresholds for each of these parameters, thereby allowing much more confident assessments to be made with respect to whether oil or gas is present.

A further advantage of this analysis is that it is possible to assess the confidence associated with analysis based on specific geochemical parameters. This allows individual parameters to be identified that have sufficiently high confidence in order to use for the fluid type analysis. Optionally, only parameters having a high confidence may be considered, with low confidence parameters not being used. Alternatively, multiple parameters may be examined (optionally including even parameters having low confidence thresholds) and a weighted analysis of their results may be used to identify a fluid type. The weightings can be based on the confidence associated with the respective region-specific threshold for each parameter.

As discussed above, whilst advanced mud-gas analysis is comparatively cheap compared to collecting a large number of downhole fluid samples, it does still represent a significant additional cost compared to collecting only standard mud-gas data. Therefore, if analysis can be performed using only standard mud-gas data, this would be highly advantageous.

Standard mud-gas data typically provides a good approximation of the C₁, C₂ and C₃ composition of the reservoir fluid. Empirically determined correction factors (usually in the range of 1-10) also exist to equate raw standard mud-gas data to the reservoir fluid composition for C₁, C₂ and C₃. Whilst these approximations are not as accurate as those that can be determined by advanced mud-gas data, they are nevertheless usable.

Standard mud-gas data typically gives poor approximations for the C₄ and C₅ compositions of the reservoir fluid. This is because, without heating of the drilling mud, only very low quantities of these components are released. Therefore, significant correction factors are required (usually in the range of 100-1000), which also amplify any noise in the measurements.

This means that, if the region-specific threshold associated with the C₁/C₂ ratio or the Bernard parameter (C₁/C₂+C₃) exhibit sufficient confidence that they can be used to confidently differentiate between oil and gas reservoir fluid for a particular field, then standard mud-gas data can be used for this analysis without the need to collect more costly advanced mud-gas data. Specifically, when considering the degree of confidence required, a greater degree of confidence is required when using standard mud-gas data than when using advanced mud-gas data due to the lower accuracy of the data itself.

Should the analysis indicate that neither of the C₁/C₂ ratio or the Bernard parameter (C₁/C₂+C₃) exhibits sufficient confidence, then the drilling operator knows that they will be required to collect advanced mud-gas data in order to accurately distinguish between oil and gas within the reservoir.

Alternatively, in situations where C₄ and C₅ data are required, a more cost-effective option than collecting advanced mud-gas data may be to instead employ a heated standard mud-gas analysis tool (or to employ a single, advanced mud-gas analysis tool on only the drilling mud outlet to collect standard mud-gas data).

Normally, standard mud-gas analysis tools do not employ heating, but some more recent tools do employ heating, such as up to about 50° C. This is lower than is normally used when collecting advanced mud-gas data, where the analysis tool typically heads the drilling mud to temperatures of up to about 90° C. However, by employing these higher temperatures (either up to about 50° C., or about 90° C.) in the collection of standard mud-gas data, it is possible to increase the quantities of C₄ and C₅ hydrocarbons released from the drilling mud. This significantly reduces the extraction efficiency correction coefficients that must be applied to the raw C₄ and C₅ measurements, and hence improves their accuracy.

If the confidence associated with the relevant geochemical properties is sufficiently high, i.e. those requiring C₄ and C₅ measurements, it may be possible to use this data for the purposed of fluid typing, even though the data is not as accurate as advanced mud-gas data.

Whilst the analysis regarding whether or not C₄ and C₅ data is required is ideally determined based on the confidences determined for the particular region, the inventors have identified that there is most commonly low confidence associated with the C₁ to C₃ geochemical parameter thresholds in situations where the reservoir gas is of high liquid yield (e.g. having a gas-oil ratio between 600 and 1200 Sm³/Sm³) and the reservoir oil is highly volatile (e.g. having a gas-oil ratio between 300 and 600 Sm³/Sm³).

Consequently, a decision regarding whether or not C₄ and C₅ data is required may be taken based on the compositions of the reservoir oil and the reservoir gas within the region of interest.

A method of identifying a fluid type of a target reservoir fluid will now be described that employs the principles above.

The first step is to determine reservoir-specific thresholds and associated confidences for a plurality of geochemical parameters.

First, a set of geochemical parameters is first identified. The geochemical parameters should be parameters that are derivable from only C₁ to C₅ fluid composition data, such that they can be derived from mud-gas data. Ideally, they would include at least one parameter derivable from only C₁ to C₃ fluid composition data, which might then permit standard mud-gas data to be used, as discussed above.

The geochemical parameters may include any combination of the C₁/C₂ ratio, the Bernard parameter (C₁/C₂+C₃), the balance ratio (C₁+C₂/C₃+C₄+C₅), the wetness ratio (C₂+C₃+C₄+C₅/C₁+C₂+C₃+C₄+C₅), the dryness ratio (C₁/C₁+C₂+C₃+C₄+C₅), and the hydrocarbon character (C₄+C₅/C₃). Other geochemical parameters may additionally or alternatively be used, such as other ratios of the C₁ to C₅ compositions, optionally treating one or more of iC₄, nC₄, iC₅ and nC₅ as separate components of that ratio.

A region of interest is then identified, i.e. where the target reservoir fluid resides. The region of interest may correspond to a specific field or basin containing the target reservoir fluids, or at least a region geographically proximate the fluid to be identified.

Next, reservoir fluid properties data for a plurality of fluid samples from a region of interest are obtained. A database containing such data will usually be available from at least initial exploratory and appraisal wells. In the case of a more mature oil fields, data will also be available from productions wells or other wells drilled within the field.

Reservoir fluid properties data represents the composition of a fluid sample from a reservoir, typically including the composition in terms of each of C₁ to C₃₆₊ hydrocarbons. Reservoir fluid properties data is sometimes referred to as PVT data because measured reservoir fluid properties data is commonly obtained in a pressure-volume-temperature (PVT) laboratory, where researchers will employ various instruments to determine reservoir fluid behaviour and properties from the reservoir samples.

Often, reservoir fluid properties data is available from a large number of wells, both within and outside of the region of interest. There are many techniques available to identify which reservoir fluid samples fall within the region of interest. For example, this may be determined based on geophysical data, from which it is possible to identify the geographic extent of a particular oil field or oil basin. Alternatively, or additionally, the properties of the reservoir fluid samples may be examined. As discussed above, the oils and gases within a particular region of interest may be, respectively, compositionally similar, which can also be used to identify if particular samples are from the same basin.

Once the reservoir fluid properties data corresponding to a plurality of fluid samples within the region of interest have been identified, the reservoir fluid properties data is parameters in order to determine a fluid type and each of the selected geochemical parameters for each of the fluid samples that are within the region of interest. This is a routine process, which will not be described in detail herein.

Next, a region-specific threshold for each of the geochemical parameters is determined based on the fluid type of the plurality of fluid samples within the region of interest. In the simplest case, this may simply comprise selecting a mid-point between the closest gas and oil parameter values. However, any suitable statistical method may be used to determine the region-specific parameter threshold.

Additionally, a confidence may be determined for each region-specific threshold, which is indicative of a confidence associated with that region-specific threshold for distinguishing between oil and gas. In the simplest case, this may be based simply on the gap between the closest gas and oil parameter values. However, again, any suitable statistical method may be used to determine the confidence.

Optionally, an assessment may then be made regarding whether to collect standard mud-gas data or advanced mud-gas data when drilling a well through the region of interest. This assessment may be made based on the confidences associated with the region-specific thresholds for geochemical parameters derivable only from C₁ to C₃ compositional data. The assessment may be made by examining individual confidences, for example requiring at least one confidence to exceed a predetermined threshold, or based on a combined analysis of multiple confidences, such that a combined confidence based on analysis of two or more such geochemical parameters exceed a predetermined threshold.

If the confidence associated with analysis based on geochemical parameters derivable only from C₁ to C₃ compositional data is sufficiently high, then optionally only standard mud-gas data may be required. If the confidence is not sufficiently high, then advanced mud-gas data may be required, and a mud logging service provider may be instructed accordingly to collect this data when drilling the well through the region of interest.

Next, mud-gas data from a well drilled through a target reservoir fluid within the region of interest is obtained. Either standard mud-gas data or advanced mud-gas data may be used, as appropriate for the relevant geochemical parameters.

This may be obtained by drilling a new well. However, it will be appreciated that the method may also be applied to historic mud-gas data from wells drilled previously, for example retrieved from a database of historic mud-gas data. Advantageously, where standard mud-gas data is appropriate for use, this technique may have wide application potentials because standard mud-gas data is always collected while drilling, whereas advanced mud-gas data is an optional service and only available when ordered, typically for exploration wells.

From the mud-gas data, the geochemical parameters of the target reservoir fluid based are calculated. Depending on the geochemical parameters to be examined, either all of the geochemical parameters having region-specific thresholds may be calculated, or only a subset of these having sufficiently high confidences.

Finally, based on the calculated geochemical parameters, the fluid type of the target reservoir fluid may be identified. This comprises comparing each of the calculated geochemical parameters against the corresponding region-specific threshold.

As will be apparent from FIGS. 4 to 8, it is expected that for geochemical parameters having a sufficiently high confidence, the analysis based on each of the geochemical parameters will agree. Where there is disagreement, a determination may be made simply based on the fluid type having the greatest number of geochemical parameters indicating that fluid type. However, in some embodiments, a weighted analysis may be performed based on the confidence associated with the threshold for each geochemical parameter.

Using this technique, it is possible to achieve a much higher accuracy when determining the fluid type of a target reservoir fluid, often using only standard mud-gas data. Testing by the inventors indicates that this technique can achieve an accuracy higher than 90% using standard mud-gas data, when applied to appropriate reservoirs.

As discussed above, this technique is advantageous because it can be performed using only mud-gas data collected when drilling a new well. Not only is this significantly cheaper than other fluid analysis techniques, but it also does not require interruption of the drilling process. Furthermore, because mud-gas data is collected as a substantially continuous log along the length of the well, it is possible to create a substantially continuous reservoir fluid type log along the length of the well. This is not possible using techniques such as downhole fluid analysis, which only identify the fluid composition at a relatively small number of target locations along the length of the well.

By using mud-gas data, the reservoir fluid type can be identified in real-time as the well is drilled. Therefore, it can be used for geosteering, which is the process of adjusting the borehole position (such as inclination and azimuth angle) as the borehole is drilled in order to reach one or more geological targets.

Furthermore, by having a continuous log, it is possible to identify thin oil-containing reservoirs that might otherwise be missed. This is particularly advantageous when applied to existing wells within a mature field, which contain wells that are considered to be dry, but do in fact still provide access to viable oil reserves.

The method described above is preferably performed by a computer program operating on a computer.

1. A method of identifying a fluid type of a target reservoir fluid within a region of interest, comprising:
obtaining reservoir fluid properties data corresponding to a plurality of fluid samples;
identifying a fluid type and at least one geochemical parameter for each of the fluid samples that are within the region of interest, the or each geochemical parameter being derivable from C₁ to C₅ fluid composition data;
determining a region-specific threshold for the or each geochemical parameter based on the fluid type of the plurality of fluid samples within the region of interest, the region-specific threshold being for distinguishing between a first fluid type and a second fluid type within the region of interest;
obtaining mud-gas data from a well drilled through the target reservoir fluid;
identifying at least one geochemical parameter for the target reservoir fluid based on the mud-gas data; and
identifying a fluid type of the target reservoir fluid based on the region-specific threshold associated with the or each of the at least one geochemical parameter for the target reservoir fluid.

2. The method according to claim 1, where the at least one geochemical parameter is comprises at least one of:
a C₁/C₂ ratio;
a Bernard parameter, C₁/(C₂+C₃);
a balance ratio, (C₁+C₂)/(C₃+C₄+C₅);
a wetness ratio, (C₂+C₃+C₄+C₅)/(C₁+C₂+C₃+C₄+C₅);
a dryness ratio, C₁/(C₁+C₂+C₃+C₄+C₅); and
a hydrocarbon character, (C₄+C₅)/(C₃).

3. The method according to claim 1, further comprising:
determining a threshold confidence for the region-specific threshold associated with the or each geochemical parameter, the threshold confidence being indicative of a confidence associated with the region-specific threshold for distinguishing between the first fluid type and the second fluid type within the region of interest.

4. The method according to claim 3, further comprising:
identifying at least one distinguishing geochemical parameter from amongst the one or more geochemical parameter based on the threshold confidences,
wherein identifying at least one geochemical parameter for the target reservoir fluid comprises identifying the at least one distinguishing geochemical parameter for the target reservoir fluid, and
wherein identifying the fluid type of the target reservoir fluid is based on the region-specific threshold associated with the or each of the at least one distinguishing geochemical parameter for the target reservoir fluid.

5. The method according to claim 3, further comprising:
determining that a threshold confidence associated with a geochemical parameter derivable from C₁ to C₃ fluid composition data is above a predetermined level,
wherein obtaining the mud-gas data comprises obtaining standard mud gas data in response to the determination.

6. The method according to claim 3, further comprising:
determining that a threshold confidence associated with a geochemical parameter derivable from C₁ to C₃ fluid composition data is below a predetermined level;
wherein obtaining the mud-gas data comprises obtaining advanced mud gas data and/or obtaining standard mud gas using heating in response to the determination.

7. The method according to claim 1, wherein the first fluid type is reservoir oil and the second fluid type is reservoir gas.

8. The method according to claim 1, wherein the region of interest comprises a single hydrocarbon field or a single hydrocarbon basin.

9. The method according to claim 1, wherein the mud-gas data comprises a plurality of mud-gas data points corresponding to target reservoir fluids at a plurality of locations along a length of the well, and wherein identifying the fluid type comprises identifying a fluid type of the target reservoir fluid at each location along the length of the well.

10. The method according to claim 1, further comprising:
determining a perforation location of a casing of the well based on the identified fluid type; and
perforating the casing of the well at the determined location.

11. The method according to claim 1, wherein:
obtaining the reservoir fluid properties data comprises reading the reservoir fluid properties data from a database; and/or
obtaining the mud-gas data comprises reading the mud-gas data from a database or receiving data input by a user.

12. The method according to claim 1, wherein at least one of the following steps is performed by a computer:
identifying a fluid type and at least one geochemical parameter for each of the fluid samples that are within the region of interest;
determining the region-specific threshold;
determining the threshold confidence;
identifying the one or more geochemical parameter for the target reservoir fluid; and
identifying a fluid type of the target reservoir fluid

13. The method according to claim 1, wherein obtaining the mud-gas data comprises measuring mud-gas data as the well is drilled.

14. The method according to claim 13, wherein the method further comprises:
steering drilling of the well based on the fluid type of the target reservoir fluid.

15. A computer program product or a tangible computer-readable medium storing a computer program product, the computer program product comprising computer readable instructions that, when executed by a computer, will cause the computer to perform a method comprising:
obtaining reservoir fluid properties data corresponding to a plurality of fluid samples;
identifying a fluid type and at least one geochemical parameter for each of the fluid samples that are within a region of interest, the or each geochemical parameter being derivable from C₁ to C₅ fluid composition data;
determining a region-specific threshold for the or each geochemical parameter based on the fluid type of the plurality of fluid samples within the region of interest, the region-specific threshold being for distinguishing between a first fluid type and a second fluid type within the region of interest;
obtaining mud-gas data from a well drilled through a target reservoir fluid;
identifying at least one geochemical parameter for the target reservoir fluid based on the mud-gas data; and
identifying a fluid type of the target reservoir fluid based on the region-specific threshold associated with the or each of the at least one geochemical parameter for the target reservoir fluid.

16. A computer comprising:
a memory, and
a processor,
the memory storing computer readable instructions that, when executed by the processor will perform a method comprising:
obtaining reservoir fluid properties data corresponding to a plurality of fluid samples;
identifying a fluid type and at least one geochemical parameter for each of the fluid samples that are within a region of interest, the or each geochemical parameter being derivable from C₁ to C₅ fluid composition data;
determining a region-specific threshold for the or each geochemical parameter based on the fluid type of the plurality of fluid samples within the region of interest, the region-specific threshold being for distinguishing between a first fluid type and a second fluid type within the region of interest;
obtaining mud-gas data from a well drilled through a target reservoir fluid;
identifying at least one geochemical parameter for the target reservoir fluid based on the mud-gas data; and
identifying a fluid type of the target reservoir fluid based on the region-specific threshold associated with the or each of the at least one geochemical parameter for the target reservoir fluid.
